(19) United States
(12) Patent Application Publication
(10) Pub. No.: US 2004/0097892 A1
(43) Pub. Date: May 20, 2004

(54) Single use urine absorbing external catheter for men

(76) Inventors: Michael Lee Evans, Wichita, KS (US); James Roy Doehla, Pleasant Hill, MO (US)

Correspondence Address: MICHAEL LEE EVANS, 7317 CEDARIDGE CIRCLE, WICHITA, KS, 67226, US

(21) Appl. No.: 10/299,922

(22) Filed: Nov. 20, 2002

(51) Int. Cl. A61F 5/44

(52) U.S. Cl. 604/349

(57) ABSTRACT

The catheter has a retainer/seal made of soft material glued to the inside circumference at its opening. The catheter is applied by inserting the penis head into the opening and pulling the catheter retainer/seal onto the penis shaft. The excess material at the opening is wrapped around the shaft of the penis and secured with a short length of tape applied to the outside of the catheter. The retainer/seal encircles the penis shaft just proximal to the penis head. The lower (distal) surface of the retainer/seal shoulders up against the ridge formed by the penis head. The diameter of the penis shaft is less than the diameter of the penis head at its proximal end. Because the retainer/seal has been fitted to the smaller diameter penis shaft, the catheter cannot be removed without deforming the penis head.

### CROSS-REFERENCE TO RELATED APPLICATIONS

| | | | |
|---|---|---|---|
| 4,601,716 | July 1986 | Smith | 604/349 |
| 4,685,913 | August 1987 | Austin | 604/349 |
| 4,790,834 | December 1988 | Austin | 604/349 |
| 5,300,052 | April 1994 | Kubo | 604/349 |
| 5,531,725 | July 1996 | Steer | 604/319 |
| 5,662,631 | September 1997 | Marx | 604/349 |
| 5,685,870 | November 1997 | Tangh | 604/349 |
| 5,695,485 | December 1997 | Duperret et al | 604/349 |
| 5,746,222 | May 1998 | Simon et al | 128/085 |
| 5,948,489 | September 1999 | Hopkins | 428/34.9 |
| 6,007,526 | December 1999 | Passalaqua et al | 604/349 |
| 6,015,550 | January 2000 | Marquis | 424/76.1 |
| 6,107,537 | August 2000 | Elder et al | 604/364 |
| 6,113,582 | September 2000 | Dwork | 604/349 |
| 6,209,142 | April 2001 | Mattsson | 2/403 |
| 6,229,062 | May 2001 | Mandell et al | 604/367 |
| 6,284,232 | September 2001 | Calton et al | 424/76.1 |
| 6,336,919 | January 2002 | Davis et at | 604/349 |

### BACKGROUND OF THE INVENTION

Holding a catheter in place on the penis has presented a challenge for numerous inventors of the past. Methods for retaining a catheter to a penis are presented in the following discussion. The following three U.S. Patents utilize a catheter retainer that is attached to the base of the penis: U.S. Pat. Nos. 6,336,919, 5,300,052, 6,113,582. When a catheter is secured at the base of the penis, the entire penis can be subjected to urine and urine vapor. The skin of the penis can suffer significant deterioration over a relatively short period of time.

Retention of a catheter by affixing it to the user's underwear is described by two U.S. Pat. Nos. 6,209,142 and 5,695,485.

Retention of a catheter by using a vacuum is described by U.S. Pat. No. 5,662,631. The vacuum retention system catheter is not a disposable catheter and would require a significant amount of effort to maintain the device and its required vacuum generating system. Retention of a catheter by using adhesives that are applied directly to the penis is described by the following four U.S. Pat. Nos. 5,531,725, 5,746,222, 4,790,834, 6,007,526. Damage to the skin of the penis can occur when removing a catheter that has been retained by adhesives. Retention of a catheter, by the application of an elastic band, is described by two U.S. Pat. Nos. 4,601,716 and 4,685,913.

Retention of a catheter by using heat-shrink plastic is described by U.S. Pat. No. 5,948,489. The penis is constricted by the elastic bands in an effort to prevent urine leakage. U.S. Pat. No. 5,685,870 discloses a unique retention system for a catheter that relies on the elasticity of the single piece catheter that envelopes the penis head. The catheter is further retained by the elasticity of the foreskin that is rolled back over the catheter to secure the catheter to the uncircumcised penis.

In addition to retaining the catheter on the penis, it is necessary to maintain the health of the skin that is in contact with the catheter. U.S. Pat. No. 6,107,537 discloses methods of incorporating skin care chemicals as part of the structure of incontinence devices.

A significant problem of incontinence is odor. Three (3) U.S. Pat. Nos. 6,284,232, 6,229,062 and 6,015,550 disclose effective chemical compounds that reduce or eliminate urine odor effectively.

### SUMMARY OF THE INVENTION

The catheter presented here is a one-use disposable device. This catheter has a waterproof shell and attaches to the penis by mechanical engagement between the penis head and the retainer/seal. The catheter is divided into an upper chamber and a lower chamber by the diagonal seal that allows a small opening between the upper and lower chambers. Urine is delivered to the lower chamber by gravity and is captured and deodorized by the chemical capsule (see FIGS. 1 and 2). The catheter is worn on the end of the penis—covering the head of the penis and about one inch of the shaft of the penis. The catheter is not designed to be used while sleeping or lying down. Urine does not flow down to the lower chamber unless the catheter is kept in a relatively vertical orientation. The catheter is to be worn between urinations. The catheter must be removed prior to urination. The penis and scrotum must be washed after removal of the catheter of prior to the application of a new catheter in order to reduce both the odor of urine and reduce the possibility infection. The catheter can be applied to any penis but the circumcised penis (lacking foreskin) provides the best structure for retaining the catheter on the penis.

### DESCRIPTION OF THE DRAWINGS

FIG. 1 shows the catheter with each component clearly labeled:

Opening

Retainer/Seal

Upper Chamber

Sealed Sides

Diagonal Seal

Lower Chamber

Chemical Capsule

Shell

Sealed Bottom

FIG. 2:

FIG. 2 is composed of 3 different drawings that show how this catheter is to be applied to a circumcised penis. FIG. 2A shows a diagrammatically bare circumcised penis. FIG. 2B shows how this catheter is applied. FIG. 2C shows how this catheter is applied to the penis and retained by wrapping the excess material around the shaft of the penis.

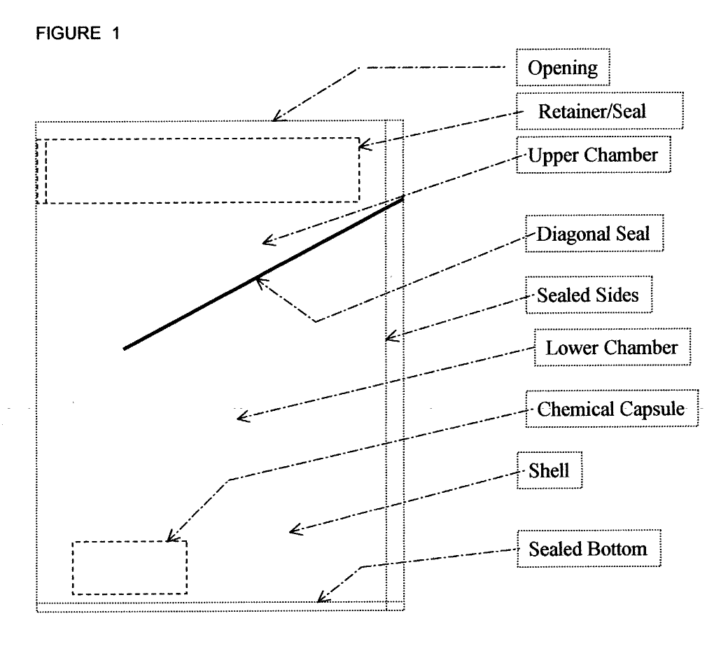

### DETAILED DESCRIPTION OF THE INVENTION

The catheter is essentially a plastic bag with a combination retainer/seal at the opening and a chemical capsule at the bottom of the bag. The catheter shell of flexible plastic is formed into a tube that has sealed sides and sealed bottom. The shell of the catheter may be made from thin waterproof plastic of polypropylene, polyvinyl, polyethylene or similar material. A diagonal seal bonds the front and back together and divides the catheter into upper and lower chambers. The diagonal seal serves to prevent the penis from protruding too far into the catheter. Both the chemical capsule and the retainer/seal contain hydrophilic and odor absorbing chemicals. Odor control in this catheter is provided by baking soda. Odor control may be provided by many chemicals and baking soda was chosen for availability and low cost. Hydrophilic material used in both the chemical capsule and the retainer/seal is sodium polyacrylate. Any hydrophilic chemical would be suitable for use in both the chemical capsule and the retainer/skin as long as it was compatible with the user's skin.

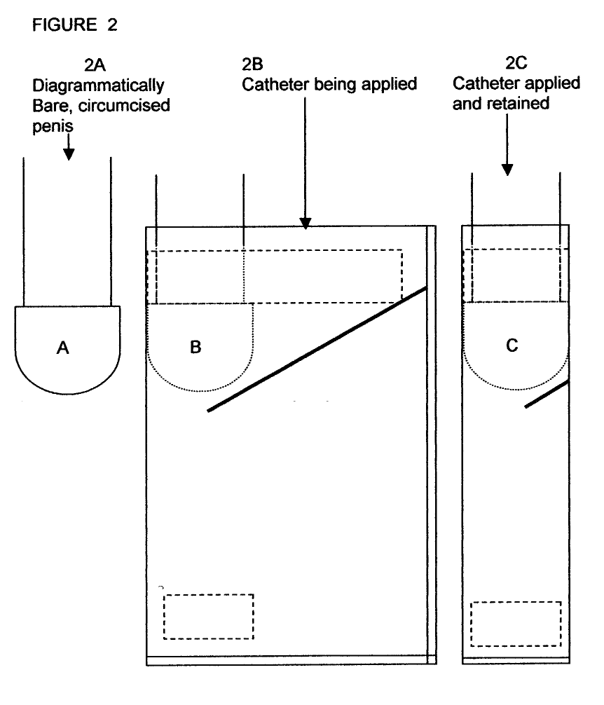

The catheter has an opening with a thin, soft, and absorbent retainer/seal attached to the inside circumference. This seal not only seals against the escape of urine and odors, but it also serves as the mechanism for retaining the catheter on the penis. The retention of the catheter is dependent on the fact that the shaft of the penis has a diameter that is smaller than the diameter of the proximal ridge of the penis head (see FIG. 2). When the penis is inserted into the catheter, the retainer/seal at the catheter opening encircles the penis shaft as the excess shell material is wrapped around the shaft. Tape applied to the outside of the catheter keeps the excess shell material snugly fit to the shaft. The snug fit of the Retainer/seal to the penis shaft provides a necessary trap for liquid and odor and it also provides the mechanism that holds the catheter to the penis. The catheter cannot be removed from the penis without distorting the penis head (see FIG. 2).

We claim:

1. The external catheter has a retainer/seal at the opening, a diagonal seal that divides the catheter into upper and lower chambers, a chemical capsule in the lower chamber, and these components are contained in a waterproof shell.

2. The retainer/seal consists of a soft and absorbent fabric glued to the inside circumference at the opening of the Catheter.

3. Both the retainer/seal and the chemical capsule contain urine absorbing chemicals and odor absorbing chemicals.
